(19) United States
(12) Patent Application Publication
(10) Pub. No.: US 2025/0082968 A1
(43) Pub. Date: Mar. 13, 2025

(54) APPARATUS AND METHOD FOR CLOSED LOOP TFUS STIMULATION

(71) Applicant: Sanmai Technologies, PBC, Sunnyvale, CA (US)

(72) Inventors: Tomokazu SATO, Minneapolis, MN (US); Timothy MULLEN, Hidden Meadows, CA (US); Christopher DAFT, Tucson, AZ (US)

(21) Appl. No.: 18/821,032

(22) Filed: Aug. 30, 2024

Related U.S. Application Data
(60) Provisional application No. 63/582,162, filed on Sep. 12, 2023.

(51) Int. Cl. A61N 7/00

(52) U.S. Cl. CPC A61N 7/00; A61N 2007/0026

(57) ABSTRACT

A transcranial Focused Ultrasound (tFUS) system uses a neural network to correct skull aberrations and maximizes the transmission of ultrasound waves through the skull. A method using supervised learning generates aberration correction parameters to be used by the receiver and transmitter of the tFUS system. A method utilizing these aberration correction parameters operating on the tFUS system maximizes the coherence of ultrasound waves passing through the skull. The method maximizes the amount of power transmitted through the skull, given a fixed maximum pressure (for example, determined by regulatory requirements).

### CROSS-REFERENCE TO RELATED APPLICATIONS

This application claims priority benefit of U.S. Provisional Application No. 63/582,162 filed Sep. 12, 2023 and entitled “APPARATUS AND METHOD FOR CLOSED LOOP TFUS STIMULATION”. The subject matter of this related application is hereby incorporated herein by reference.

### BACKGROUND

### Field of the Various Embodiments

This invention relates to trans-cranial focused ultrasound systems (tFUS) and in particular to closed-loop tFUS stimulation, wherein feedback in various forms is used to improve the efficacy of a tFUS session.

The efficacy of tFUS systems may be enhanced by guiding the stimulation beam using closed-loop feedback. The purpose of these closed-loop methods is to maximize the therapeutic effect of the ultrasound stimulation which reaches the target structure. There is a need in the art for alternative feedback methods that reduce cost and provide additional treatment capabilities.

### BRIEF DESCRIPTION OF THE DRAWINGS

FIG. 1A illustrates a prior art tFUS application including an FUS apparatus positioned inside of a magnetic resonance (MR) head coil and a corresponding mannequin image.

FIG. 1B illustrates a prior art acoustic energy profile (longitudinal and transversal) with associated acoustic parameters.

FIG. 2 is a graph presented in a paper by Wynn Legon et al, including group average FFT (fast Fourier transform) EEG (electroencephalographic) frequency spectra, shown as normalized power vs frequency with a Sham and tFUS response.

FIG. 3 illustrates a low-intensity tFUS system using neuronavigation presented in a paper by Young Goo Kim et al.

FIG. 4 illustrates a tFUS setup having an EEG sensor array coupled to the subject's head, with the output fed back to control the stimulation beam, in an embodiment of the present disclosure.

FIG. 5 illustrates a tFUS setup similar to FIG. 4 wherein evoked potential pattern recognition and/or a phase-locked-loop may be inserted into the feedback loop, in embodiments of the present disclosure

FIG. 6 illustrates the phase-locked-loop of FIG. 5, in an embodiment of the present disclosure.

FIG. 7 illustrates a version of the tFUS setup of FIG. 4 wherein feedback from an electrooculography (EOG) sensor or from a electromyography (EMG) sensor is included, in embodiments of the present disclosure.

FIG. 8 illustrates a tFUS setup including a magnetic resonance imaging (MRI) system in an embodiment of the present disclosure.

FIG. 9 illustrates a tFUS setup including a functional near-infra-red spectroscopy (fNIRS) system, in an embodiment of the present disclosure.

FIG. 10 illustrates a tFUS setup wherein ultrasonic measurements of blood flow or blood properties are employed in the feedback loop, in accordance with an embodiment of the present disclosure.

FIG. 11 is a flow diagram illustrating a method for generating efficacy information using tFUS.

### DETAILED DESCRIPTION

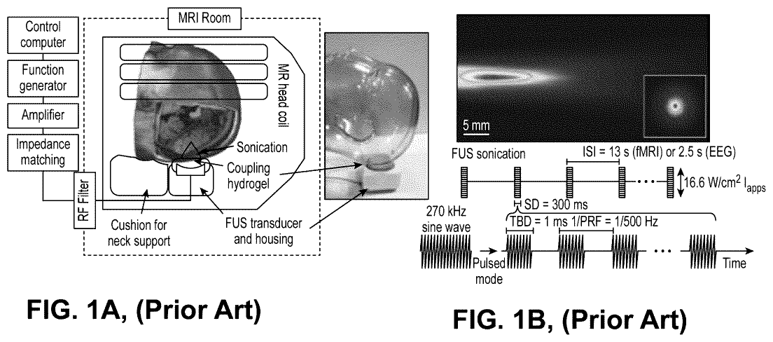

FIGS. 1A and 1B are presented in a paper by Wonhye Lee et al. FIG. 1A illustrates a prior art tFUS application including an tFUS apparatus positioned inside of a magnetic resonance (MR) head coil, together with a corresponding mannequin image. The tFUS apparatus includes an transducer and housing, a coupling hydrogel, a sonication region, and an MR head coil. A computer controls a function generator, an amplifier and an impedance matching network that connects through an RF filter to the tFUS transducer.

FIG. 1B illustrates a prior art acoustic energy profile (lateral and axial) with associated acoustic parameters SD (sonication duration), ISI (inter-stimulation interval), together with TBD (tone burst duration) and PRF (pulse-repetition frequency).

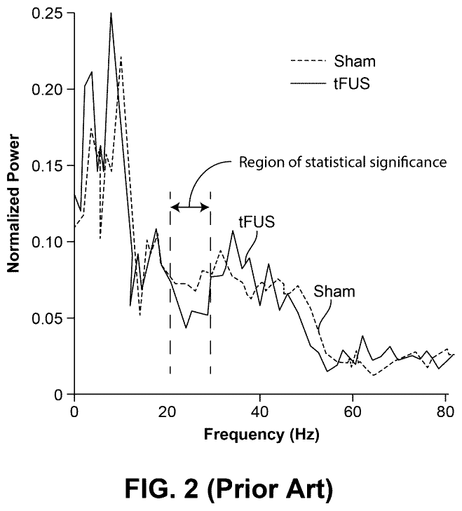

FIG. 2 is a graph presented in a paper by Wynn Legon et al, including group average FFT (fast Fourier transform) frequency spectra, shown as normalized power vs frequency with a Sham and tFUS response. A region of statistical significance is shown. The Sham response is the response achieved when the stimulation beam is turned off, but all other measurement parameters remain unchanged.

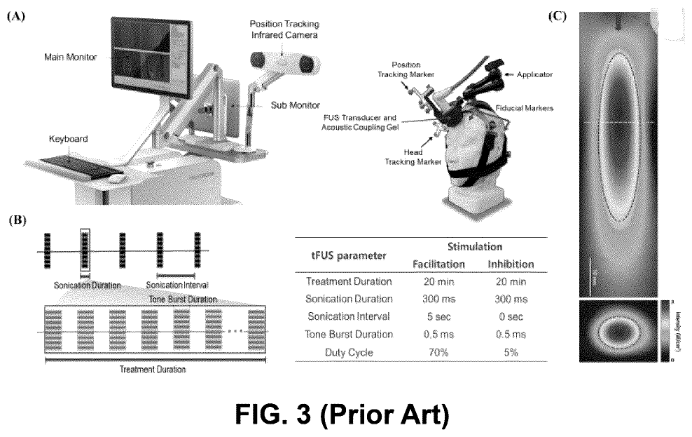

FIG. 3 illustrates a low-intensity tFUS system presented in a paper by Young Goo Kim et al, including a PC, monitor, infra-red cameras, and headgear in group (A), a schematic diagram of sonication parameters in group (B), and an acoustic intensity profile in lateral and axial planes in group (C).

Embodiments of the present disclosure will now be discussed. Transcranial focused ultrasound can act as a neural stimulus. Systems and methods for closed loop tFUS neuromodulation will be described, wherein tFUS stimulation characteristics are controlled by physiological measurements or functional imaging techniques, at least in part.

One simple approach to brain stimulation is to push neuronal activation up or down as far as possible in an open-loop fashion. Embodiments described herein are more useful with respect to clinical or non-clinical well-being by adjusting parameters to achieve a healthy range; the closed-loop stimulation enables finely controlled modulation of brain activity.

Many types of illness are inadequately treated by existing methods. Embodiments described herein can improve patient care through reduction in anxiety (for example, Generalized Anxiety Disorder), reduction in depression (for example, Major Depressive Disorder), and inhibition of progression of Alzheimer's disease.

The transcranial focused ultrasound approach has advantages over pharmacological interventions because of reduced side-effects and improved efficacy. It has advantages over non-drug methods such as Transcranial Magnetic Stimulation (TMS) because ultrasound can affect deeper structures and can require fewer treatment sessions. Ultrasound can also be focused and steered by mechanical or electronic means.

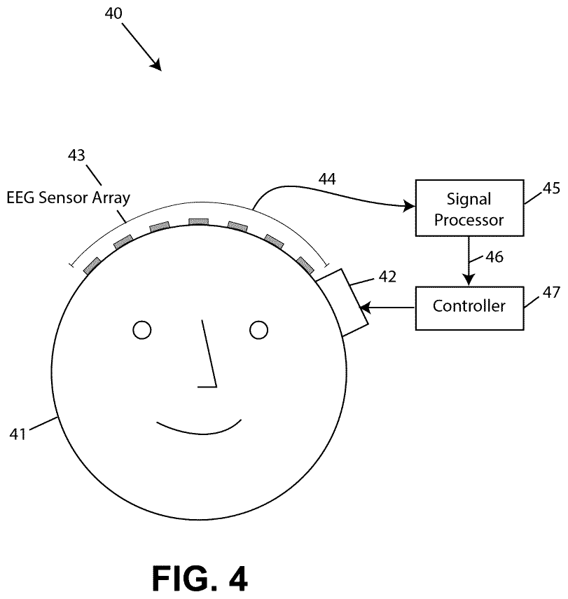

FIG. 4 illustrates a basic tFUS setup 40 wherein a subject's head 41 receives stimulation from an ultrasound transducer 42 while an attached electroencephalography (EEG) sensor array 43 is sensing responses 44. Signal processor 45 applies basic signal processing functions such as amplification and Fast Fourier Transformation (FFT) to identify brainwaves in distinct frequency bands such as Beta, Alpha, Theta, and Delta bands. These processed signals are fed 46 to controller 47 which applies adaptive controls to the ultrasound transducer 42 to complete the feedback loop. EEG data provides feedback that can be used to design scientifically informed strategies to reduce stress, improve focus or enhance meditation. Abnormal EEG data can indicate signs of brain dysfunction, head trauma, sleep disorders, memory problems, brain tumors, stroke, dementia, seizure disorders like epilepsy and various other conditions.

Three special types of EEG signals are used in embodiments of the present disclosure: evoked potentials caused by tFUS (one-shot responses to the ultrasound, produced 1-200 ms after the ultrasound stimulation), evoked potentials generated by external stimuli or internally by the subject and modulated by tFUS, and oscillations seen in EEG from one or more locations in the brain (i.e., frequencies found over a longer period).

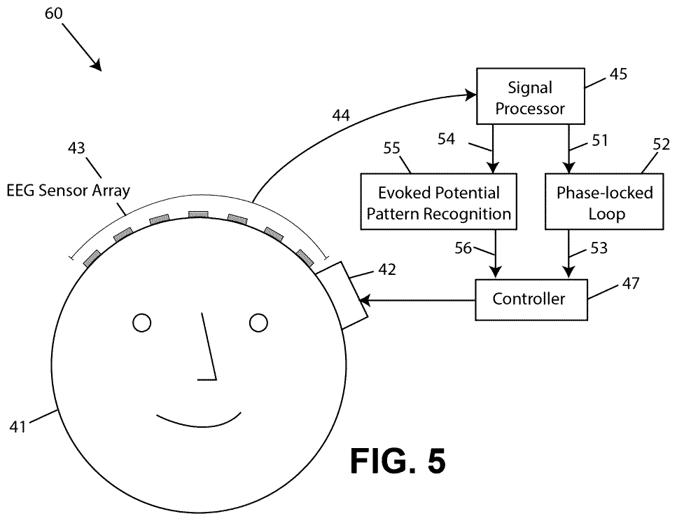

tFUS-evoked potentials are useful for enabling plasticity changes that can change the connectivity between two brain regions. These plasticity changes between two neurons occur when there is a specific timing of their action potentials relative to each other (as first postulated in the concept of Hebbian learning (“neurons that fire together, wire together”). With this approach, a group of neurons is stimulated by tFUS at two different times. Effective synchronization between the two regions is achieved by monitoring the evoked potentials recorded by EEG. This technique can be used to either couple (upstream area fires first, immediately followed by downstream areas) or decouple groups of neurons (typically by the opposite timing, random firing in noise patterns, or lack of other correlated timing). FIG. 5 illustrates additional processing options including a branch 51 employing a phase-locked loop 52, and a branch 54 including evoked potential pattern recognition 55. In addition, for two connected regions that are being modulated by such timing-dependent stimulation protocols, one can also have test trials where the upstream region only is stimulated by tFUS of a known intensity (i.e. evoked potential caused there). One can then measure the evoked potential of the downstream region and assess whether the connection between these two regions have been strengthened or weakened properly. Such trials can be interspersed at regular or random intervals within the primary timing-dependent stimulation protocol.

tFUS-evoked potentials may also be used in a more direct method even for a single brain region. In one example a subject may be asked to attempt to move a limb upon a known timing cue. In addition, there may be a change in EEG signals from the relevant brain areas (seen either as an evoked potential or change in spectral power of oscillations). Based on that cue (to internally generate a specific neural state or volitional signal) or changes in EEG, a tFUS stimulus to elicit an evoked potential in the motor cortex may be delivered. This motor example may be applicable to those who lost motor function due to stroke or have motor issues due to Parkinson's, but the general concept applies to other scenarios of pairing a cued, internally generated neural state or signal with a tFUS-evoked potential. Another similar example could be to deal with addiction. While a typical target may be the dopaminergic areas, an alternative could use negative valence brain regions such as the amygdala or portions of the habenula. One may ask a subject to imagine the sight, smell, and taste of cigarettes, or even present visual and olfactory cues relating to smoking, while inducing evoked potentials with tFUS in brain regions coding negative valence. The external signal could also be downstream of the tFUS-evoked potential. A simple example again would be the tFUS stimulation of the motor cortex. However, this time, the external stimulus is the electrical or magnetic stimulation of corresponding muscle groups or the motor neurons innervating them.

Modulation of evoked potentials, such as those generated by internal imagination, volition, or recollection, as well as those caused by external stimuli, is another critical feedback signal. Fear, PTSD, startle, and related negative valence emotions or disorders of such emotions will be used for an instructive example. In this case, a sudden sound, a cue or sensory stimulus that can trigger PTSD, or a command to start recollecting a negative or traumatic memory may be given. Upon these cues, evoked potentials corresponding to activation in various brain regions may be seen. We may expect signals changes in the amygdala or the posterior cingulate cortex. One may apply tFUS in an anti-Hebbian manner as discussed previously. However, alternatively, one may attempt to modulate these cue-evoked potentials by applying an inhibitory waveform to relevant brain areas to lessen the effects as well. Repeated exposure of these negative stimuli can be used, potentially as part of more conventional cognitive or exposure therapy, or eye movement desensitization and reprocessing (EMDR) therapy. Ultrasound may also be used to elicit the eye movements for EMDR, effectively by causing an evoked potential in motor regions controlling the eyes.

EEG oscillations, commonly analyzed for their power spectra and phase of various frequency components, have long been used in neuroscience to measure mental states.

These oscillations may be used in a similar manner as evoked potentials as briefly alluded to earlier. Instead of a noticeable evoked potential, a change in oscillation characteristics (amplitude in specific frequencies, increase or decrease of oscillation frequencies, etc.) may be seen in response to cues, commands, sensory stimuli, or internal volition and recall. The changes will often be transient and can be repeatedly measured by giving these cues after breaks. The changes in these oscillation changes in response to tFUS can be used for feedback.

In an embodiment the tFUS stimulation is phase-locked using phase-locked-loop 52 with a periodic component of the EEG signal. An example of use is to search for peaks in the power spectrum and then phase lock the stimulation to the EEG received from a certain location in the brain. Existing algorithms can be used to localize EEG signals. Once the ultrasound is phase locked to the neural activity, it can be used to shift the magnitude, phase, and frequency of this portion of the EEG signal.

As a specific example, a stimulatory tFUS waveform may be applied during the rising phase of an EEG oscillation to speed up the frequency or magnitude of the signal at the frequency. Alternatively, an inhibitory tFUS waveform may be applied to delay the oscillation peak or magnitude.

In another embodiment branch 54 and evoked potential pattern recognition 55 of FIG. 5 are utilized to analyze EEG signals via power spectra. EEG spectra exhibit several strong peaks since much neural activity is periodic. In an EEG spectrum, peak locations in frequency space and the size of the spectral peaks yield important information such as their position within the EEG bands, as described by Gavaret et al (see references).

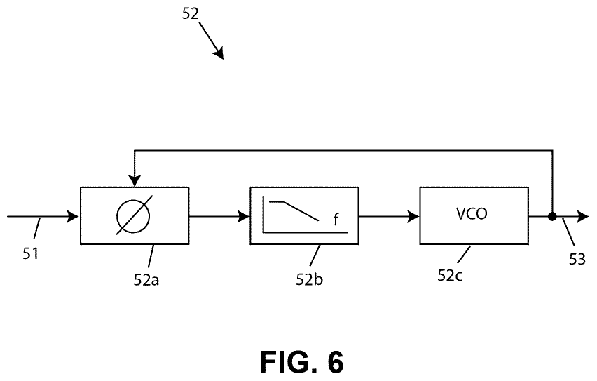

FIG. 6 illustrates a phase-locked-loop 52 employed in embodiments of the present disclosure. The EEG signal processed by signal processor 45 feeds into a phase comparator 52a which feeds a low-pass filter 52b and a voltage-controlled oscillator 52c. The output 53 is fed back to the phase comparator 52a and feeds forward to the controller 47. The components of phase-locked-loop 52 may be either analog or digital components. The phase lock enables localization of the periodic component in both space and frequency.

EEG oscillations from two brain regions may also be used as a feedback signal. Two regions that are connected will often have correlated phases and changes in oscillation characteristics. Similar to the discussed example of analyzing the magnitude and delays of evoked potentials in an area downstream of a tFUS-stimulated brain area, one can use these correlations as a measure of the connectivity between two brain regions. A simple example might be that increases in amplitude or decrease in frequency of oscillations in a certain frequency band in one brain region may be known to affect a downstream region. One may use Hebbian-like stimulation at these two brain regions to alter connectivity. Alternatively, one may simply enhance certain oscillation characteristics in both regions (i.e. cause them both to have more power in a specific frequency band; or to adjust their relative phases to be closer or further apart in time, or even to decorrelate or decouple their phases). Changes in correlations and coupling in the oscillations between the two regions, during periods without tFUS, could be used as a signal for feedback in such cases. Similarly, the MRI technique known as diffusion tensor imaging (DTI) can potentially be used to assess connectivity changes in a feedback system.

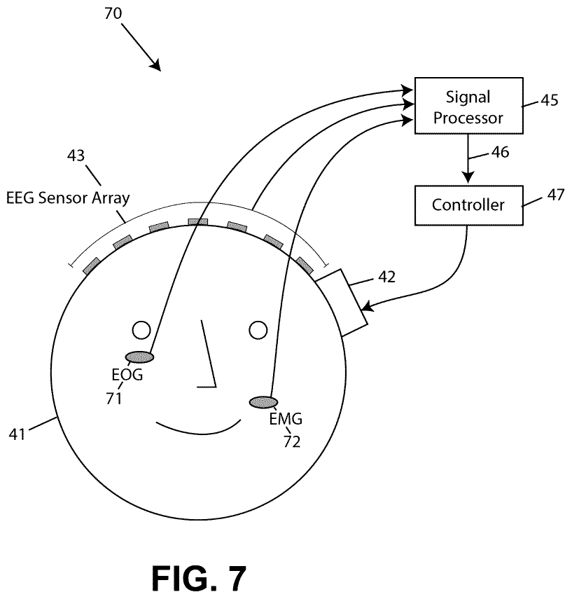

FIG. 7 introduces the use of two special sensors, an electrooculography (EOG) sensor and an electromyography (EMG) sensor, in embodiments of the present disclosure. The EOG sensor measures the potential difference between the retina and the cornea of a human eye, which can be modeled as a dipole with a positive potential on the cornea and a negative potential on the retina. By attaching skin electrodes outside the eye, the potential can be measured by having the patient move the eyes horizontally. The EMG sensor detects electrical activity from a muscle using conductive pads placed on the skin. Electrical impulses cause fibers within a muscle to contract. These two new forms of feedback can be correlated with EEG data within signal processor 45 to inform exploratory procedures, with potential efficacy for patient care.

EMG can be used as feedback improving the efficacy of tFUS. A simple example would be the stimulation of the correct parts of the motor cortex should lead to modulation of EMG. This may be useful in athletic (improvement of force output or synchrony of motor neuron activity) or rehabilitative (injury or degenerative disease) scenarios. One may stimulate the motor cortex while asking the patient to move a specific limb or muscle to enhance coordination between the brain and body, the mind and muscle.

EMG and EOG can be useful as well in the previously discussed case of fear, startle, or PTSD treatment as they can be used as proxies of reflexive movements. Reduction of these responses can be used as a sign that the tFUS is modulating the correct brain targets with effective waveforms. In addition, other physiological readouts such as heart rate, galvanic skin responses, breathing patterns (such as rate and depth), O2/CO2 ratios in breath, and pupil size and fixation can be used as physiological feedback to adjust tFUS in a closed loop fashion.

EMG, EOG, and physiological measurements can be used in at least two ways. First, a brain region of interest, when modulated, may be expected to lead to changes in EMG, EOG, and similar physiological measurements. In this case, these changes can be used to both validate the targeting of the ultrasound field, including for adjustments to aberration correction, and to adjust the waveform parameters to improve efficacy, reliability of evoking changes, or amplitude/magnitude of changes. Secondly, even if the target brain region, upon modulation, is not expected to lead to direct changes in these recordings, if there are “test” regions that have similar neural responses (as known from animal or human literature), the waveform parameters can be adjusted again for efficacy using these “test” regions. In addition, if the relative spatial arrangement between the target and “test” regions are consistent across people, they can also be used for improved aberration correction and triangulation.

In one such embodiment, the controller, along with any relevant software on the controller or to interface with it, would receive information about the intended target brain region and type and polarity of modulation desired by the clinician or the user. The controller will then determine parameters to test as well as options of other “test” brain regions to aid in waveform optimization or spatial targeting if such approaches are more suitable. The controller will then use the starting ultrasound parameters for neuromodulation while recording the physiological measurements and observing the size and sign of any changes. Using EMG, EOG, etc., the controller will then assess if the results seen in response to test ultrasound waveforms are as expected from previous trials of the same subject or from population level data. If the results are larger than desirable, the waveform duration and intensity, for example, can be lowered by the controller. Alternatively, with lower than expected results, the controller can direct the ultrasound beam around the area to see if there is a more responsive location due to possible incomplete aberration correction or individual anatomical differences. In addition, the waveform intensity or duration can be extended. Other parameters such as phase and frequency of tone bursts can also be adjusted to seek improvements.

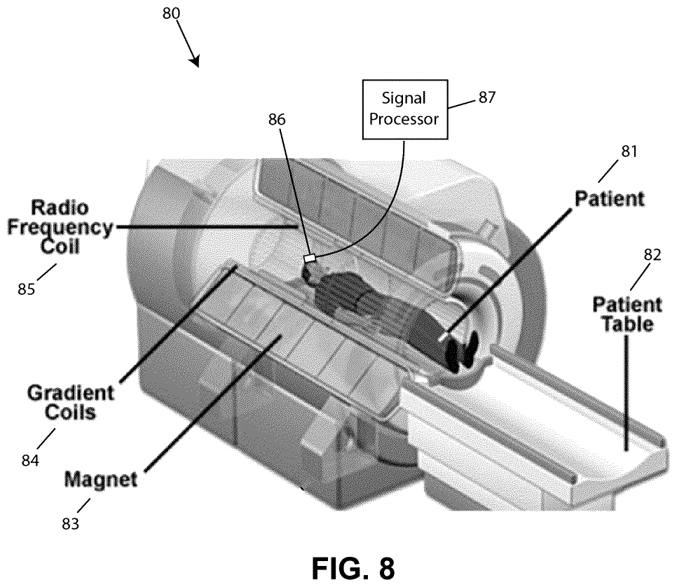

FIG. 8 illustrates a tFUS setup 80 that includes Functional MRI (fMRI), in an embodiment of the present disclosure. fMRI measures brain activity by detecting changes associated with blood flow. This technique relies on the coupling of cerebral blood flow and neuronal activation: when an area of the brain is in use, blood flow to that region increases. fMRI has been used to study psychiatric disorders including schizophrenia, bipolar disorder, autism spectrum disorders, and attention deficit hyperactivity disorders. It is generally an expensive procedure, with a typical cost exceeding $500 per session. FIG. 8 shows a patient 81, a patient table 82, a magnet 83, gradient coils 84, a radio frequency coil 85, an ultrasonic transducer 86, and a signal processor 87.

In an embodiment blood oxygen level dependent (BOLD) contrast is an example of fMRI used to verify that targeting of the ultrasound stimulus is correct by assessing hemodynamic changes in neuronal oxygen consumption. It can also determine whether the activity is changing in the desired way.

Arterial Spin Labeling (ASL), wherein inflowing blood is magnetically labeled by a radiofrequency pulse generated by the scanner, can provide similar information to BOLD. ASL can be used to confirm that a desired target was hit without requiring a behavioral result, such as might be measured using a questionnaire. In another embodiment fMRI is used to monitor both skull temperature and soft tissue, to verify that the tFUS treatment is benign.

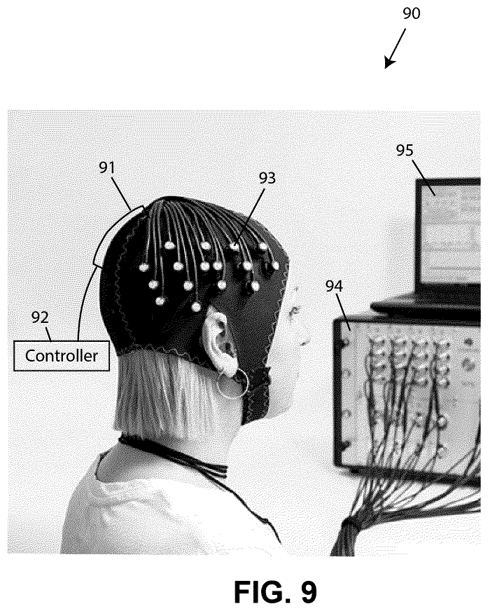

In an embodiment functional near-infrared spectroscopy (fNIRS) provides data similar to BOLD data (arguably today's gold standard) but it is limited to superficial structures; also, its resolution can be poorer than fMRI. A related technique is time-domain NIRS (TD-NIRS). FIG. 9 illustrates a tFUS setup 90 using fNIRS in an embodiment of the present disclosure. An ultrasonic transducer 91 creates ultrasonic stimulation under control of controller 92. Feedback is provided by an array of devices 93 arranged on the subject's head, wherein each device transmits a near-infra-red (NIR) signal and measures an NIR response. A controller 94 for the NIR signals is shown. Sensed NIR signals are displayed on the monitor 95.

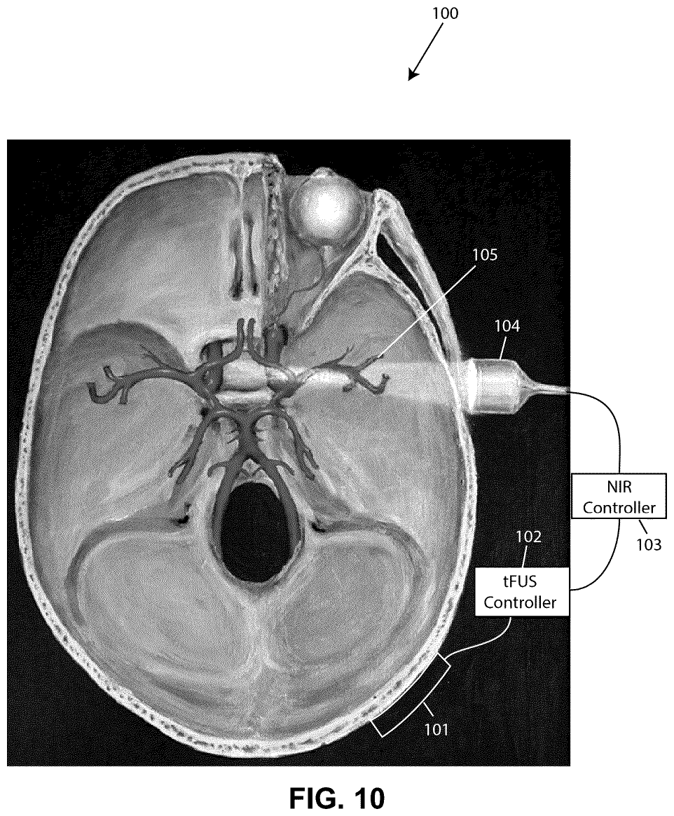

Other data acquisition methods may be used to close the feedback loop. In ultrasound perfusion imaging, Color Doppler Mode is used to produce feedback in the form of Doppler shifted ultrasound; this method has been used to assess perfusion in the brain for more than two decades. FIG. 10 illustrates a tFUS setup 100 that includes Color Doppler Mode in an embodiment of the present disclosure. An ultrasonic transducer 101 is controlled by a tFUS controller 102 which shares information with an NIR controller 103. Controller 103 controls NIR transceivers 104. Blood vessels 105 are shown. In addition, recent advances in localization microscopy have enhanced the ability of diagnostic ultrasound to resolve small vessels and low flow rates, improving perfusion data quality.

Other techniques to measure blood flow, blood volume, or tissue physical characteristics due to the change in metabolism or blood perfusion (elastographic methods using ultrasound are common for this), and changes in neurotransmitter concentrations can similarly be used instead of evoked potentials or changes in oscillations to indicate changes in brain activity. These may include blood flow measurement and imaging techniques such as RF and microwave-based imaging, volumetric impedance phase shift spectroscopy (VIPS), or electrical impedance tomography (EIT) which have been successfully used in stroke detection. In addition, spectroscopic methods such as magnetic resonance spectroscopy can be used to measure the amount or the change in the amount of specific neurotransmitters in brain regions. Such signals can also be used as a proxy of whether a brain region has potentially increased activity (increase in glutamate) or decreased (decrease in glutamate or increases in GABA or glycine).

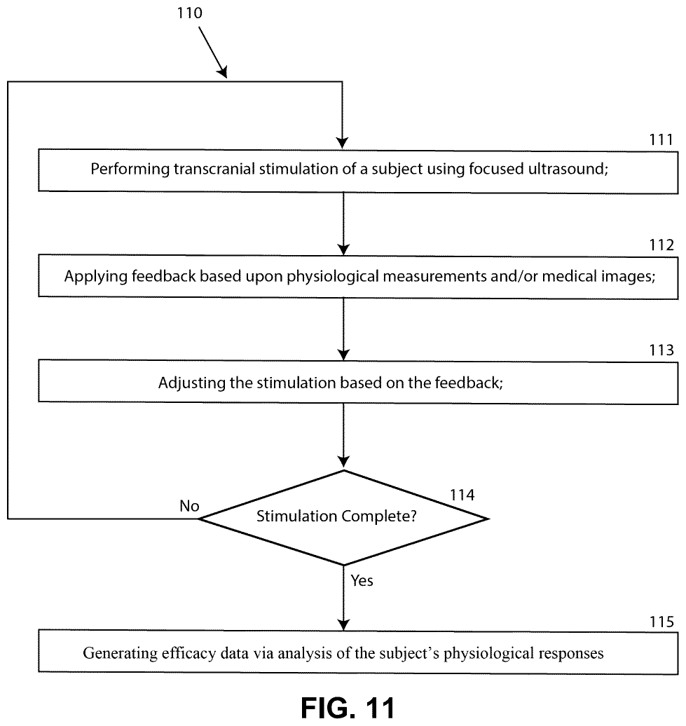

FIG. 11 describes a method 110 for generating efficacy information using tFUS, in an embodiment of the present disclosure. The method comprises: performing transcranial stimulation of a subject using focused ultrasound, step 111; applying feedback based on physiological measurements and/or images, step 112; adjusting the stimulation based on the feedback, step 113; checking to see if the stimulation is complete, step 114; and finally, generating efficacy data via analysis of the subject's physiological responses if the stimulation is complete, step 115. In an embodiment, the application of feedback or the adjustment of the stimulation or analysis of the subject's physiological responses is enhanced using machine learning (ML) or artificial intelligence (AI).

Using physiological data (EEG, MEG, EMG, EOG, fNIRS, etc.) one can generate data regarding the efficacy of the ultrasound waveform and targeting used for the individual subject. The efficacy can be quantified in a number of ways. These include but are not limited to magnitude of evoked potentials; strength of inhibition of activity or incoming potentials; modulation of evoked potentials; changes to the frequency, phase, or amplitude of brain region oscillations; strength of coherence between connected areas; blood flow or other metabolic or hemodynamic measures of brain activity induced by the ultrasound pulse. Analyzing these factors in comparison to previous data from the individual as well as population-level data, the controller can adjust parameters including but not limited to: aberration correction; aiming/targeting; overall ultrasonic field extent and shape; timing parameters of the ultrasound waveform such as PRF, especially of frequency ranges relevant to brain activity (<10 kHz); phase of such lower frequency waveform components; amplitude of the waveform; duration of the waveform.

In the embodiments of the present disclosure the collection of physiological signals, as described in reference to FIGS. 4-11, is performed using a single subject session. In each case selected physiological signals are used to modify the stimulation during the subject session.

The DMN is a separate set of brain regions that exhibits strong low-frequency oscillations coherent during resting state and is thought to be activated when individuals are focused on their internal mental-state processes, such as self-referential processing, interoception, autobiographical memory retrieval, or imagining future. The negative effects of rumination may be studied using the DMN. In an embodiment, the methods described herein are applied to disruption of the DMN.

As the DMN constitutes a strongly connected network as described before, measurements such as coherence and oscillation frequency can be analyzed. As mentioned before, application of ultrasound and the corresponding changes to these parameters can be used to assess strength of modulation. To improve the strength of ultrasonic neuromodulation, one can raster the beam around to find a better location to account for individual anatomical differences or increase the waveform intensity or duration. In addition, using the EEG data, a better matching phase and frequency waveform could be applied to the subject. In addition, using ultrasound-evoked potentials (activation), one could see if these pulses are strongly transmitted to other regions using EEG or other physiological brain activity recording methods. From there, as before, the controller can be used to make adjustments to various ultrasonic parameters. Such methods allow for quantified and objective measurements of the effects of ultrasonic neuromodulation, offer with many more trials or much more data, and hence more rapid adjustments, than relying purely on qualitative feedback such as mood, especially since qualitative feedback can be heavily influenced by other factors.

What is claimed is:

1. A transcranial ultrasound device comprising:
an ultrasound transducer, configured to transmit stimulation signals;
an electroencephalography sensor array, wherein the ultrasound transducer receives feedback in response to the stimulation signals; and
a controller configured to adjust at least one subsequent stimulation signal in response to the feedback received by the electroencephalography sensor array.

2. The system of claim 1, wherein the ultrasound transducer, the electroencephalography sensor array, and the controller are configured in a closed loop configuration.

3. The system of claim 1 wherein the stimulation signals are phase-locked to a periodic signal measured by the electroencephalography sensor array.

4. The system of claim 1, wherein the controller causes the ultrasound transducer to utilize evoked potentials in the stimulation signals in response to the feedback in response to the stimulation signals obtained by the electroencephalography sensor array.

5. The system of claim 1 wherein the feedback is derived from a plurality of brain regions.

6. The system of claim 1 wherein the feedback comprises modulation of evoked potentials.

7. The system of claim 1, wherein the controller is further coupled to an electrooculography (EOG) sensor.

8. The system of claim 1, wherein the controller is further coupled to an electromyography (EMG) sensor.

9. The system of claim 1, wherein the ultrasound transducer stimulates a selected brain region, and the feedback is derived from physiological signals or medical imaging data are obtained during a single subject session by the electroencephalography sensor array.

10. The system of claim 9, wherein the physiological signals are used to modify the stimulation signals during the subject session.

11. The system of claim 9, wherein the selected brain region is the amygdala.

12. The system of claim 9, wherein the selected brain region is the posterior singulate cortex (PCC).

13. The system of claim 1 wherein the feedback is further obtained via a functional magnetic resonance imaging (fMRI) device.

14. The system of claim 13 wherein the fMRI comprises Blood Oxygen Level Dependent (BOLD) contrast.

15. The system of claim 13 wherein the fMRI comprises Arterial Spin Labeling (ASL) contrast.

16. The system of claim 1 wherein the feedback is derived from functional near infra-red spectroscopy (fNIRS).

17. The system of claim 1 wherein the feedback is derived from Doppler ultrasound.

18. A method comprising:
performing transcranial stimulation of a subject using ultrasound using an ultrasound transducer;
obtaining feedback in response to the transcranial stimulation via a electroencephalography sensor array; and
generating, via a controller, an adjustment to the transcranial stimulation based upon the feedback obtained by the electroencephalography sensor array.

19. The method of claim 18, further comprising adjusting the transcranial stimulation based upon at least one of physiological measurements or medical imaging data.

20. The method of claim 18 wherein generating the adjustment of the transcranial stimulation or analysis of the subject's physiological responses is enhanced using machine learning or artificial intelligence.

21. The method of claim 18, further comprising generating efficacy data based upon an analysis of the physiological measurements or medical imaging data.

22. The method of claim 19, further comprising disruption of the Default Mode Network (DMN).
